(19) United States
(12) Patent Application Publication
(10) Pub. No.: US 2011/0264091 A1
(43) Pub. Date: Oct. 27, 2011

(54) APPARATUS AND METHOD FOR SEALING SPECIMEN FOR RETRIEVAL

(76) Inventors: Rachel Suzanne Koppleman, Cambridge (GB); Charlotte Adele Clark, Cambridge (GB); Timothy N. Wells, Manchester, CT (US); John Leach, Woodbridge, CT (US); Denese Ann-Marie Slowly, Milford, CT (US); Alistair Ian Fleming, Cambridge (GB); Nicholas John Collier, Cambridge (GB)

(21) Appl. No.: 13/080,780

(22) Filed: Apr. 6, 2011

Related U.S. Application Data
(60) Provisional application No. 61/327,850, filed on Apr. 26, 2010.

(51) Int. Cl. A61B 18/14; A61B 17/32

(52) U.S. Cl. 606/41; 606/167

(57) ABSTRACT

A method for modifying a tissue specimen includes providing a specimen modifying apparatus configured to modify the specimen to thereby prevent seeding from the specimen. The specimen modifying apparatus is inserted through an opening in tissue and positioned adjacent the tissue specimen to be modified. The tissue modifying apparatus is activated to modify the tissue specimen to thereby inhibit seeding from the tissue specimen.

This application claims priority from provisional application Ser. No. 61/327,850, filed Apr. 26, 2010, the entire contents of which are incorporated herein by reference.

### BACKGROUND

1. Technical Field

The present disclosure relates to specimen retrieval and, more particularly, to apparatus and methods for minimizing the risk of seeding during specimen retrieval.

2. Background of Related Art

Laparoscopic and endoscopic surgical procedures are minimally invasive procedures in which operations are carried out within the body by using elongated instruments inserted through small entrance openings in the body. The initial opening in the body tissue to allow passage of the endoscopic or laparoscopic instruments to the interior of the body may be a natural passageway of the body, or it can be created by a tissue piercing instrument such as a trocar. Laparoscopic procedures generally require that any instrumentation inserted in the body be sealed, i.e. provisions must be made to ensure that gases do not enter or exit the body through the instrument or the entrance incision so that the surgical region of the body, e.g. the peritoneum, may be insufflated. Mechanical actuation of such instruments is for the most part constrained to the movement of the various components along a longitudinal axis with structure provided to convert longitudinal movement to lateral movement where necessary.

Because the endoscopic or laparoscopic tubes, instrumentation, and any required punctures or incisions are relatively narrow, endoscopic or laparoscopic surgery is less invasive as compared to conventional surgical procedures in which the surgeon is required to cut open large areas of body tissue. Therefore, laparoscopic or endoscopic surgery minimizes trauma to the patient and reduces patient recovery time.

Minimally invasive procedures may be used for partial or total removal of body tissue or organs from the interior of the body, e.g. nephrectomy, cholecystectomy, and other such procedures such as minimally invasive thoracic procedures. During such procedures, it is common that a cyst, tumor, or other affected tissue or organ must be removed via the access opening in the skin, or through a cannula. In many of these procedures, especially where cancerous tumors are removed, removal of the specimen in an enclosed environment is highly desirable to prevent seeding.

Accordingly, various types of entrapment devices have been disclosed to facilitate the removal of specimens. For example, U.S. Pat. No. 5,037,379 to Clayman et al. discloses a surgical tissue bag for percutaneously debulking tissue by morcellation. The bag includes a layer of puncture-resistant material, a layer of moisture-resistant material and a drawstring. In a disclosed method of use, the bag is placed within the body cavity, the body tissue or organ is placed within the bag, the opening of the bag is pulled through the incision in the skin leaving the distal end of the bag containing the tissue or organ within the body cavity, a morcellator is then inserted into the bag, and then the tissue or organ is debulked and suctioned out of the bag.

U.S. Pat. No. 5,074,867 to Wilk discloses a planar membrane having filaments attached to its corners. The membrane is placed within a body cavity with the filaments extending through the trocar cannula to the outside of the body. The organ or tissue to be removed is placed on the membrane and the filaments are pulled to close the membrane around the organ and draw it through the cannula, if the organ is sufficiently deformable. If the organ is not sufficiently deformable, e.g. because of the presence of gallstones, a forceps or other instrument is used to crush the stones or tissue.

Improvements to prior art entrapment devices are disclosed in U.S. Pat. No. 5,647,372 to Tovey et al. and in U.S. Pat. No. 5,465,731 to Bell et al., the disclosures of which are hereby incorporated by reference in their entirety.

It would be advantageous to provide a method and apparatus for tissue specimen removal which reduces the risk of seeding and minimizes tissue damage during removal.

### SUMMARY

The present disclosure provides apparatuses and methods for modifying a tissue specimen in such a way as to prevent seeding during removal. The present disclosure is also related in some aspects to an access port for use during retrieval of a tissue specimen from inside a patient's body. The present disclosure is applicable in conjunction with an entrapment device, or in place of an entrapment device, both embodiments being configured to prevent seeding and tissue damage during specimen removal.

In accordance with one aspect of the present disclosure, a method for modifying a tissue specimen includes providing a specimen modifying apparatus configured to modify the specimen thereby preventing the specimen from seeding upon removal from a body cavity. The specimen modifying apparatus is inserted through an opening in tissue and positioned adjacent the tissue specimen to be modified. The specimen modifying apparatus is then activated to modify the tissue specimen thereby inhibiting the tissue specimen from seeding. The method may also include retrieving the tissue specimen.

In one embodiment, the method includes positioning an access port within the opening in tissue. The tissue modifying apparatus is then inserted through the access port. The specimen may also be retrieved, or removed, through the access port.

In some embodiments, the access port can include a tubular body defining a longitudinal axis. The access port is configured for positioning within an opening in tissue and has a proximal end and a distal end. In some embodiments, a proximal radial flange extends radially outwardly from the proximal end of the tubular body and is configured to extend along an exterior surface of tissue adjacent the opening in tissue. The access port can be used for passage of the various specimen modifying apparatus described herein, as well as used for passage of other instrumentation.

In one embodiment of the present disclosure, the specimen modifying apparatus includes a housing having an elongated shaft extending from a distal end thereof. The shaft can include a lumen therein for the passage of tissue sealant therethrough and is configured for insertion through an opening in tissue. A dispensing assembly can be disposed at a distal end of the shaft and in communication with the lumen of the shaft. The dispensing assembly in one embodiment is in the form of a nozzle positionable adjacent a tissue specimen and configured to selectively dispense tissue sealant onto a surface of the specimen to seal the specimen, inhibiting the passage of cellular material between the tissue specimen and surrounding tissue. The tissue sealant may be a fibrin glue.

In accordance with another embodiment of the present disclosure, the specimen modifying apparatus includes a housing having an elongated shaft disposed at a distal end thereof and connected to a radiation source. The shaft is insertable through an opening in tissue. An end effector assembly is disposed at a distal end of the shaft and is positionable adjacent a tissue specimen. The end effector assembly can include a pair of insulated cups moveable from an open position to a closed position. The cups preferably form a closed cavity in the closed position to retain the tissue specimen therein. The cavity in one embodiment can be spherical. The cups are preferably insulated to minimize the passage of radiation therethrough, and upon activation, radiation energy is supplied from the radiation source to the tissue specimen retained within the cups to terminate the cells of the tissue specimen with radiation. The cups may be lead-lined to minimize the passage of radiation through the cups.

In another embodiment of the present disclosure, the specimen modifying apparatus includes a housing having an elongated shaft disposed at a distal end thereof, the housing configured to operably couple to a source of electrical energy. An end effector assembly is disposed at a distal end of the shaft and is positionable at least partially through an opening in tissue. The end effector assembly can include a pair of electrically conductive plates spaced apart from one another and configured for positioning a tissue specimen therebetween. Preferably electrical energy is selectively applicable from the electrical energy source to the pair of electrically conductive plates to thereby pass a voltage through the specimen. The voltage passed between the electrically conductive plates may in some embodiments be about 4 kV. The voltage passed through the specimen may be above a threshold, thereby resulting in nonreversible electroporation, or termination of cells therein. Alternatively, the voltage passed through the specimen may be below the threshold, thereby resulting in electroporation of the cells to permit permeation therethrough of fluids which would otherwise be impermeable.

In another aspect, a specimen modifying apparatus is provided comprising a housing having an elongated shaft extending from a distal end thereof. The shaft includes a lumen therein for the passage of tissue sealant therethrough and is configured for insertion through an opening in tissue. A dispensing assembly is disposed at a distal end of the shaft and in communication with the lumen of the shaft, the dispensing assembly positionable adjacent a tissue specimen and configured to selectively dispense tissue sealant onto a surface of the tissue specimen to thereby seal the tissue specimen, inhibiting passage of cellular material between the tissue specimen and surrounding tissue.

In another aspect, a specimen modifying apparatus is provided comprising a housing having an elongated shaft disposed at a distal end thereof, the housing being connected to an energy source. The shaft is insertable through an opening in tissue. An end effector assembly is disposed at a distal end of the shaft. The end effector assembly is positionable adjacent a tissue specimen and includes a pair of members moveable from an open position to a closed position, wherein, upon activation, energy is supplied from the energy source to the tissue specimen retained within the cups to modify the tissue specimen for removal from a body.

In some embodiments, the energy source is a source of radiation and the end effector assembly includes a pair of insulated cups which form a closed cavity in the closed position to thereby retain the tissue specimen therein, the cups being insulated to inhibit the passage of radiation therethrough.

In some embodiments, the energy source is a source of electrical energy and the end effector assembly includes a pair of electrically conductive plates spaced apart from one another and configured for positioning with a tissue specimen disposed therebetween, wherein electrical energy is selectively applicable from the electrical energy source to the pair of electrically conductive plates to thereby pass a voltage through the specimen to modify the tissue specimen.

### BRIEF DESCRIPTION OF THE DRAWINGS

Various embodiments of the subject instrument are described herein with reference to the drawings wherein:

FIG. 1 is a side, cross-sectional view of an access port according to the present disclosure shown disposed through an opening in tissue;

FIG. 2 is a side view of a dispensing assembly of a specimen modifying apparatus of the present disclosure shown coating a tissue specimen;

FIG. 3 is a perspective view of a specimen modifying apparatus according to another embodiment of the present disclosure;

FIG. 4 is an enlarged view of an end effector of the specimen modifying apparatus of FIG. 3 in an open position;

FIG. 5 is an enlarged view of the end effector of the specimen modifying apparatus of FIG. 3 in a partially closed position;

FIG. 6 is a perspective view of a specimen modifying apparatus according to another embodiment of the present disclosure; and

FIG. 7 is an enlarged view of the end effector assembly of the specimen modifying apparatus of FIG. 6 shown having a tissue specimen disposed therebetween.

### DETAILED DESCRIPTION

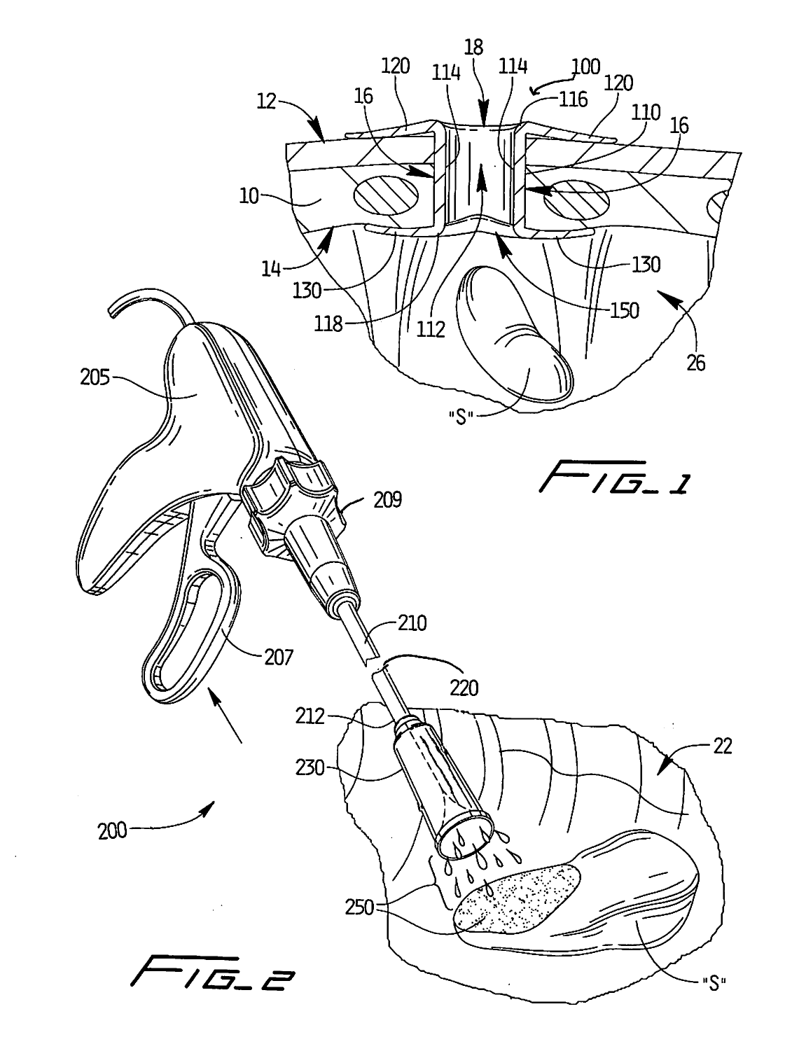

Turning now to FIG. 1, an access port 100 is shown generally including a tubular body 110 defining a longitudinal axis, a proximal flange 120, and a distal flange 130. As shown in FIG. 1, access port 100 is positionable through an opening 18 in tissue 10 to provide access to internal body cavities and/or tissue. More particularly, tubular body 110, defining a lumen 112 therethrough, is disposed through opening 18 in tissue 10 such that a wall 114 defining lumen 112 of tubular body 10 abuts surface 16 of tissue 10 defining opening 18 in tissue 10. Proximal flange 120 extends radially outwardly from proximal end 116 of tubular body 110 along and adjacent to an external surface 12 of tissue 10. Similarly, distal flange 130 extends radially outwardly from distal end 118 of tubular body 110 along and adjacent to an internal surface 14 of tissue 10.

Access port 100 may in some embodiments also include a seal 150 disposed within tubular body 110 to seal off and prevent the exchange of fluids between internal body cavities or tissues and the external environment. As can be appreciated, when access port 100 is positioned within opening 18 in tissue 10, access port 100 lines the opening 18 in tissue 10 and the immediate surrounding external 12 and internal 14 surfaces of tissue 10. Seal 150, if provided, prevents fluid exchange through opening 18 in tissue 10.

It is envisioned that access port 100 may be formed of flexible material and be compressible to fit within an opening before expanding towards an uncompressed size. Further, the access port 100 may be dimensioned according to the diameter and thickness of tissue 10 through which access port 100 is to be inserted. More specifically, tubular body 110 of access port 100 may have a diameter substantially equal to a diameter of the opening 18 in tissue 10 such that tubular body 110 is securely positionable within opening 18 in tissue 10. Similarly, tubular body 110 of access port 100 may have a length that is substantially equal to the thickness of tissue 10 such that proximal and distal flanges 120 and 130 are securely positionable on the external 12 and internal 14 surfaces of tissue 10, respectively. In other words, it is envisioned that access port 10 be dimensioned to fit securely within an opening 18 in tissue 10, held in place by the forces acting on access port 100 by surrounding tissue.

It is further envisioned that surgical instrumentation (see FIGS. 2-7) for modifying and/or retrieving a tissue specimen “S” may be inserted and removed through access port 100. Upon retrieval, tissue specimen “S” may be removed from the body through the access port 100. Accordingly, access port 100 is configured to facilitate the removal of tissue specimen “S,” while reducing the risk of damaging tissue and/or tissue seeding. Access port 100 may be made from a flexible material to conform to opening 18 in tissue 10 and/or to reduce the force on specimen “S” during retrieval.

With continued reference to FIG. 1, proximal flange 120 and distal flange 130 extend a sufficient distance radially away from opening 18 in tissue 10 so as to provide an apron around the opening 18 in tissue 10 on both the external 12 and internal 14 surfaces of tissue 10. As will be described in more detail hereinbelow, proximal flange 120 and distal flange 130 help prevent contact between specimen “S” and external and internal surfaces 12 and 14, respectively, of tissue 10 during removal of specimen “S” from inside the body. Tubular body 110 similarly prevents contact between the specimen “S” and wall 16 of tissue 10 forming opening 18 in tissue 10 during removal therethrough. Providing this barrier between specimen “S” and tissue 10 helps prevent seeding during retrieval of specimen “S.” This is especially important due to the increased contact, manipulation and forces acting on specimen “S” during retrieval, all of which increase the risk of tissue damage and/or seeding during retrieval.

The operation of access port 100 will now be described in detail with reference to FIG. 1. Initially, access port 100 is inserted through an opening 18 in tissue 10. Distal flange 130 may be bent or deformed during insertion such that distal flange 130 may pass through opening 18 in tissue 10 to an internal side 14 of tissue 10. With access port 100 inserted through opening 18 in tissue 10, tubular body 110 is disposed through opening 18 in tissue 10 such that lumen 112 provides access to internal tissue 26. Proximal flange 120 extends from tubular body 110 along external surface 12 of tissue 10, while distal flange 130 extends from tubular body 110 along an internal surface 14 of tissue 10. Surgical instrumentation, e.g., a specimen modification apparatus (see FIGS. 2-7), may then be inserted through lumen 112 of access port 100 to manipulate, modify and/or retrieve a tissue specimen “S.” Seal 150 may optionally be provided, depending on the minimally invasive surgical procedure and is configured to accept surgical instrumentation therethrough and to seal therearound such that the sealed internal environment is maintained. Specific embodiments of specimen modifying apparatuses will be described in detail hereinbelow, although other instruments not described herein are also contemplated for use with access port 100.

In order to modify and/or remove a tissue specimen “S” from inside a patient's body, a surgical instrument (not shown) is inserted through access port 100. The surgical instrument may then be activated to modify or otherwise treat the specimen. When the specimen is a cancerous mass, or tumor, for example, it would be advantageous to seal the specimen or terminate the cell mass prior to removal to prevent seeding. Once the specimen has been modified, e.g., sealed or terminated, a surgical instrument is used to enclose, grasp, suction, or otherwise retain specimen “S” thereon. The instrument is then translated proximally to remove the specimen “S” from the body via the opening 18 in tissue 10. During removal of a relatively large specimen, the relatively narrow opening 18 in tissue 10 may contact the specimen “S,” causing tearing of tissue, detachment of the specimen “S” from the surgical instrument and/or seeding. Modification of the specimen prior to removal reduces the risks of seeding. The access port 100 aids in the removal process and protects against the potential for seeding from tissue that has not been effectively modified.

Further, during removal, the specimen “S” may become misaligned with the access port and/or may be too wide to pass through the opening 18 in tissue 10 without the use of force. In such situations, as specimen “S” is translated toward the opening 18 in tissue 10, specimen “S” may brush internal surface 14 of tissue 10 and/or wall 16 defining opening 18 of tissue 10. This tissue on tissue brushing may cause damage to either tissue and/or may result in seeding. However, with the presence of access port 100 disposed through opening 18 in tissue 10, specimen “S” contacts distal flange 130, rather than internal surface 14 of tissue upon misalignment. The configuration of distal flange 130 provides a less abrasive surface such that the potential for tissue damage and/or seeding is reduced. Additionally, distal flange 130 acts to funnel specimen “S” toward lumen 112 defined within tubular body 110 of access port 100, thereby facilitating removal of specimen “S.” A lubricant (not shown) may also be applied to the inner surface of wall 114 of access port 100 to facilitate removal of the specimen “S.”

Once specimen “S” is translated into lumen 112 of tubular body 110, access port 100, in part due to its flexibility, allows specimen “S” to pass proximally though lumen 112 of tubular body 110 with minimal forces acting on specimen “S”. The relatively smooth surfaces of tubular body 110 help prevent specimen “S” from catching or tearing during translation through access port 100. As specimen “S” is translated proximally past proximal end 116 of tubular body 110, proximal flange 120 of access port 100 acts to prevent tissue damage and/or seeding during the emergence of specimen “S” from access port 100. More specifically, if specimen “S” had been deformed during passage through tubular body 110, specimen “S” may reform, e.g. extend radially outwardly, to its original shape upon exiting tubular body 110. However, proximal flange 120 prevents contact of reforming specimen “S” with an external surface 12 of tissue 10.

Thus, specimen “S” may be removed from inside a patient's body through a relatively narrow opening 18 in tissue 10 without contacting the exterior surface 12, interior surface 14, or internal wall 16 of tissue 10 surrounding opening 18 in tissue 10. This configuration helps prevent seeding, tissue damage, and specimen damage without requiring bagging the specimen for removal.

With reference now to FIG. 2, one embodiment of a specimen modifying apparatus 200 is shown. Specimen modifying apparatus in this embodiment is in the form of a tissue sealant applicator 200 and generally includes a housing 205 including at least one control member 207 and an elongated shaft 210 defining a longitudinal axis and extending from a distal end of the housing 205. The housing 205 may be configured to facilitate gripping and/or manipulation by a user. The control member is shown in the form of a trigger, but can be configured as other actuation mechanisms e.g., joystick, for dispensing tissue sealant 250, manipulating the specimen modifying apparatus 200 and/or changing the configuration of the specimen modifying apparatus 200, as will be discussed in detail below. A nozzle (or dispensing) assembly 230 is disposed at a distal end 212 of the elongated shaft 210 and is configured to selectively apply tissue sealant 250 to a tissue specimen “S.” A lumen 220 is defined through the elongated shaft 210, extending into the nozzle assembly 230 for supplying tissue sealant 250 to the nozzle assembly 230 for dispensing sealant. The control member 207 may be configured to control the dispersion of tissue sealant 250 from the nozzle assembly 230. Rotation knob 209 can rotate shaft 210 and nozzle assembly 230.

Elongated shaft 210 is insertable through an opening in tissue such that dispensing assembly 230 may be positioned adjacent a tissue specimen “S” to be modified, or sealed. Further, elongated shaft 210 may be flexible and/or steerable for improved access to tissue. The nozzle assembly 230 may be rotatable and/or radially deflectable with respect to the longitudinal axis to provide a larger coverage area for dispensing tissue sealant 250. A control mechanism, e.g., joystick, may be provided on the housing 205 for controlling the manipulation of the nozzle assembly 230. This is especially helpful where the opening in tissue is small, thereby limiting the radial movement of shaft 210 of tissue sealant applicator 200 when disposed through the opening in tissue. Additionally, nozzle assembly 230 may include a plurality of settings, e.g., solid stream, rain drops, pulsed stream, spray, etc., for selectively dispensing tissue sealant 250. For example, a solid stream setting may be chosen where the specimen to be sealed is relatively small and/or is positioned adjacent other tissue. On the other hand, a spray setting may be preferred where the specimen is relatively large and/or remote from other tissue.

Tissue sealant 250 may be any suitable tissue sealant capable of sealing and isolating a tissue specimen “S.” Such tissue sealants 250 may include, but are not limited to fibrin glues, gelatin matrix/thrombin sealants, cyanoacrylate sealants, bovine serums and polyethylene glycol hydrogels. Fibrin glues are advantageous in that they are made from biological materials, seal relatively quickly, and present a low risk of site inflammation or infection. More recent developments allow fibrin sealant to be made from the patient's own blood, thus lowering the risk of allergic reaction and/or rejection. As will be described in more detail below, nozzle assembly 230 is used to apply tissue sealant 250 to a tissue specimen “S,” i.e. to coat tissue specimen “S” with tissue sealant 250, to thereby seal specimen “S” in an isolated environment, such that cellular materials may not pass between the tissue specimen “S” and the surrounding environment 22. As can be appreciated, sealing the tissue specimen “S” inhibits seeding during the retrieval process without damaging the integrity of the tissue specimen “S.”

The operation of tissue sealant applicator 200 will now be discussed in detail with reference to FIG. 2. Initially, elongated shaft 210 of tissue sealant applicator 200 is inserted through an opening in tissue (or an access port 100 disposed through an opening in tissue) such that nozzle assembly 230 is generally directed toward a tissue specimen “S” to be retrieved, as shown in FIG. 2. Manipulation of nozzle assembly 230, e.g., radial deflection and/or rotation, may be required at this point to direct nozzle assembly 230 toward specimen “S,” depending on the location of specimen “S” relative to the opening in tissue. Next, upon activation of the control member e.g., by switching an activation switch “on” or by depressing a trigger (e.g. trigger 207), tissue sealant 250 is urged through lumen 220 of shaft 210 and into nozzle assembly 230. The specific configuration and/or setting, e.g., solid stream, pulsed stream, rain drops, spray, etc.; of nozzle assembly 230 disperses tissue sealant 250 toward specimen “S” as desired. Rotation and/or deflection of nozzle assembly 230 at this point allows tissue sealant 250 to be applied to the exposed surface of specimen “S.” A manipulation instrument (not shown) either disposed on or used in conjunction with tissue sealant applicator 200 may be used to manipulate specimen “S” to expose any un-coated portions thereof. For example, a manipulation instrument, e.g. a grasper, can be inserted through a separate access port or opening to manipulate the tissue specimen. Accordingly, tissue sealant applicator 200 may then be activated once again to complete coating of the entire surface of specimen “S” to seal specimen “S.”

Once specimen “S” has been coated with tissue sealant 250, tissue sealant applicator 200 may be removed from the opening in tissue. At this point, a surgical instrument may be inserted through the opening in tissue to grasp or otherwise retrieve the modified/treated specimen “S” therefrom. Although specimen “S” may in some embodiments be placed in a receptacle, e.g., a bag, prior to removal, this is not required because it is sealed. That is, the coating of tissue sealant 250 provides protection to specimen “S” and surrounding tissue, isolates specimen “S” thereby preventing seeding, and aids in the removal process by providing a more uniform, smooth outer surface on specimen “S.” Additionally, after specimen “S” has been sealed with tissue sealant 250, a lubricant may be applied to the sealed surface of specimen “S” to further facilitate the removal of specimen “S.” The lubricant (not shown) may be applied from tissue sealant applicator 200, e.g., via a separate lumen extending to nozzle assembly 230, or from a new instrument (not shown) inserted into the body cavity through an access port or body opening. Further, the access port 100, as mentioned above, may be similarly coated on an interior surface with a lubricant to facilitate removal of the specimen “S.” As such, relatively large specimens may be removed through relatively small openings in tissue without the need for a receptacle.

Further, access port 100 may aid in the removal of the sealed specimen “S.” More particularly, the surface features of access port 100 and sealed specimen “S” may be configured to allow for smooth translation of sealed specimen “S” through lumen 112 of access port 100, thereby facilitating removal from the body. Lubricant added to the sealed specimen “S” and/or the inner surface of wall 114 of access port 100 may also aid in the removal of the sealed specimen “S,” as mentioned above.

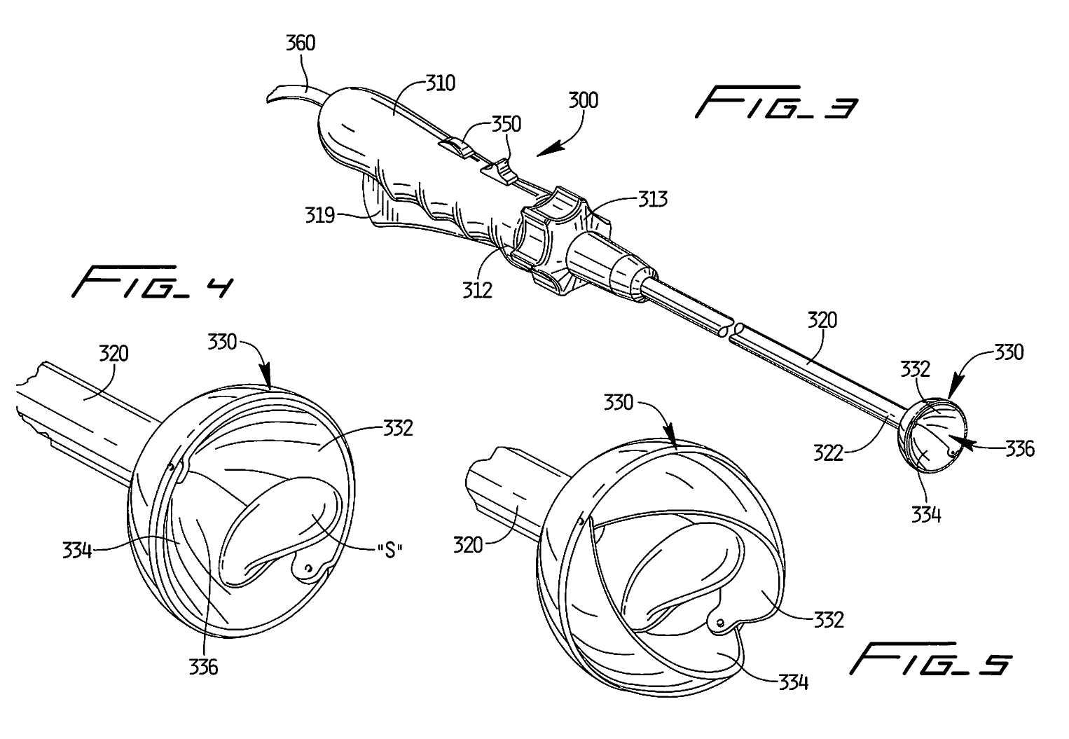

Turning now to FIGS. 3-5, another embodiment of a specimen modifying apparatus 300 is shown. Specimen modifying apparatus in this embodiment is in the form of a radiation device 300 which generally includes a housing 310 connected to a source of energy (not shown) via a cable 360. An elongated shaft 320 extends from a distal end 312 of the housing 310 and includes an end effector assembly 330 disposed at a distal end 322 thereof.

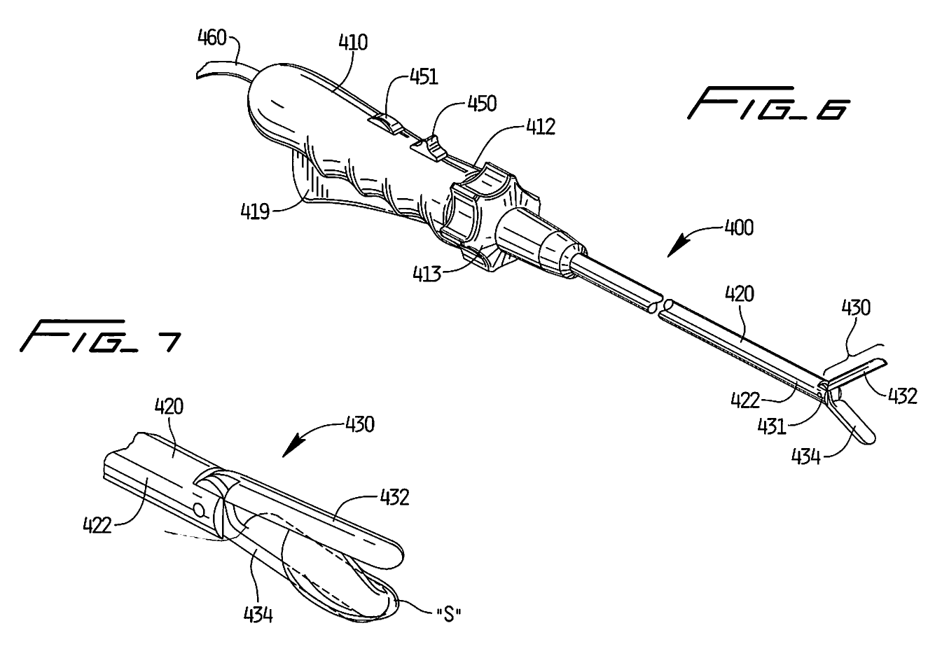

End effector assembly 330 includes a pair of opposed cups 332, 334 movable between an open position wherein cups 332, 334 are substantially overlapping with one another to form a open, partial-sphere cavity 336 (FIG. 4) to a closed position wherein cups 332, 334 are moved with respect to each other (FIG. 5) to form a fully enclosed sphere having a hollow interior, by movement of actuator 319. Cable 360, e.g., a fiber optic bundle, extending through housing 310 and shaft 320, is configured to provide radiation energy to the end effector assembly 330, e.g. via a laser or other radiation device. Cups 332, 334 may be lead lined, or otherwise insulated, to minimize the passage of radiation therethrough. In other words, when cups 332, 334 are in the closed position, radiation supplied to end effector assembly 330 is retained within the hollow sphere formed by cups 332, 334. Further, end effector assembly 330 may be rotatable together with shaft 320 by rotation of knob 313, rotatable with respect to shaft 320 and/or deflectable with respect to shaft 320, e.g., via one of control members 350 linked to actuation wires extending through shaft 320 and operably connected to end effector assembly 330, to facilitate positioning of end effector assembly 330 as required for a particular procedure.

With continued reference to FIGS. 3-5, shaft 320 is configured and dimensioned to be positionable through an opening in tissue such that end effector assembly 330 is disposed adjacent a tissue specimen “S.” As will be described in more detail below, cups 332, 334 are configured to surround a tissue specimen “S” and close therearound such that tissue specimen “S” is disposed within the hollow sphere of end effector assembly 330. Once tissue specimen “S” is retained within cups 332, 334, gamma radiation energy may be supplied to cups 332, 334 to thereby modify the specimen “S” by terminating the cells making up tissue specimen “S”, e.g. the malignant cells. Since cups 332, 334 are insulated, the radiation is contained within hollow sphere formed by cups 332, 334 and thus does not damage surrounding tissue or organs. The specimen “S” may then be removed from the body by the apparatus 300 or other conventional instrumentation. As with the previous embodiment, while a receptacle may be provided, a receptacle is not required for removal of specimen “S” since the terminated tissue is no longer capable of seeding. Alternatively, the specimen “S” may simply be left inside the body to break down.

The operation of endoscopic radiation device 300 will now be described in detail with reference to FIGS. 3-5. Prior to deployment, cups 332, 334 are initially disposed in the closed position to avoid collecting tissue and/or debris upon passage into an opening in tissue. With cups 332, 334 in the closed position, radiation device 300 may be inserted through an access port or directly through an opening in tissue lead by end effector assembly 330. Radiation device 300 is positioned such that end effector assembly 330 is adjacent a tissue specimen “S.” Next, the user may move cups 332, 334 from the closed position to the open position by actuator 319 and further advance radiation device 300 such that cups 332, 334 surround specimen “S.” The user may then move cups 332, 334 back to the closed position to retain specimen “S” therein. Radiation may then be selectively applied to the end effector assembly 300 via one of the control members 350, thereby passing through the specimen “S” disposed within cups 332, 334. As stated above, the radiation kills or terminates the cells, e.g. “disarms” the malignant cells, within cups 332, 334 but the lead-insulated cups 332, 334 prevent radiation from harming surrounding tissue disposed outside of sphere 336. Once the cells within specimen “S” have been terminated, modified/treated specimen “S” may be removed along with radiation device 300, without the risk of seeding. Alternatively, since the cells have been terminated, specimen “S” may be left inside the body of the patient to be harmlessly absorbed.

It is also envisioned that cups 332, 334 can be retractable from a substantially linear configuration to a spherical configuration shown in FIGS. 3-5. More specifically, cups 332, 334 may each be formed from a plurality of sections (not shown) which are moveable between an overlapping position and an extended position. In the overlapping position, the diameter of cups 332, 334 would be relatively small in comparison to the diameter of cups 332, 334 in the extended position. This would allow cups 332, 334 to be passed through a relatively small incision to treat a relatively large tissue specimen. In other words, the diameter of the sphere 336 formed by cups 332, 334 may be larger than a diameter of the opening in tissue since the sphere 336 is not completely formed until the end effector assembly 330 has passed through the opening in tissue. Thus, the specimen can be modified or treated using a minimally invasive procedure without the difficulties associated with removing a large specimen from a relatively small incision in tissue. Once the specimen has been treated, it may be removed intact, removed in piecemeal, or may be left behind, as mentioned in previous embodiments.

Referring now to FIGS. 6-7, yet another embodiment of a specimen modifying apparatus 400 is shown. Specimen modifying apparatus, or electroporation device 400 is shown generally including a housing 410 connected to a source of energy (not shown) via a cable 460 and an elongated shaft 420 extending from a distal end 412 of the housing 410. An end effector assembly 430 is disposed at a distal end 422 of the shaft 420. Control member(s) 450 may be used to selectively apply a voltage to the end effector assembly 430. Actuator 419 opens and closes the end effector assembly 430.

End effector assembly 430 includes a pair of opposed electrically conductive members, e.g. plates 432, 434, electrically coupled to the energy source (not shown). As will be described in more detail below, end effector assembly 430 and shaft 420 are configured for insertion through an access port or opening in tissue such that electrically conductive plates 432, 434 are disposable on opposing sides of a tissue specimen “S.” End effector assembly 430 may be rotatable together with shaft 420 by rotation knob 413, rotatable with respect to shaft 420 and/or deflectable with respect to the shaft 420, e.g. via control knob 451 linked to cables connected to end effector support 431 which can be pivotably attached (not shown) to the end of shaft 420. Electrical energy from the energy source may be provided to the end effector assembly 430 to pass a voltage between the plates 432, 434, and thus through the tissue specimen “S” disposed therebetween. When the voltage passing through the tissue specimen “S” reaches a specific threshold, which is dependent on the size and characteristics of the specimen disposed between the plates 432, 434, the voltage begins to terminate the cells making up the specimen “S.” A sufficient voltage passed between plates 432 and 434 may be about 4 kV, for example. This process of passing a voltage through tissue to kill the cells therein is generally known as irreversible electroporation. Irreversible electroporation is advantageous in that, while the tissue specimen “S” disposed between the plates 432, 434 is killed, surrounding tissue and organs are left unharmed. Alternatively, a lower voltage may be provided to effect reversible electroporation. Reversible electroporation involves sending a specific voltage through cellular membrane to create micropores therein. The micropores allow fluids to pass therethrough that would otherwise be impermeable through an unmodified cell membrane. As in previous embodiments, once the specimen “S” is modified/treated, the specimen “S” may be removed from the body, or may simply be left therein.

The operation of electroporation device 400 will now be described in detail with reference to FIGS. 6-7. Initially, elongated shaft 410 of electroporation device 400 is inserted at least partially through an opening in tissue or access port with plates 432, 434 in an approximated (closed) position to ease insertion. Actuator 419 is activated to separate the plates 432, 434 and then the plates 432, 434 are disposed on either side of a specimen “S” to be modified/treated. From this position, a voltage, e.g., 4 kV, may be passed from plate 432 through tissue to plate 434 to terminate all cells in specimen “S” disposed between the plates 432, 434. As mentioned above, the voltage may be sufficiently large to terminate all cells therein, thereby effecting nonreversible electroporation. Alternatively, a voltage below the nonreversible electroporation threshold may be applied to allow fluids to pass into the cells making up the specimen, as mentioned above. In such an embodiment, fluids may be provided to terminate or otherwise modify the specimen as desired. Once all cells of the specimen “S” have been modified or terminated, the specimen “S” can be removed or simply left behind as described above with regard to the discussion of the embodiment of FIG. 3.

The instruments described above can be used in minimally invasive procedures such as laparoscopic surgery and thoracic surgery, e.g. VATS (video assisted thoracic surgery), where access to the thoracic cavity is achieved less invasively through the intercostal space (between adjacent ribs).

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A method for modifying a tissue specimen comprising the steps of:
providing a specimen modifying apparatus configured to modify the specimen to prevent seeding from the specimen;
inserting the specimen modifying apparatus through an opening in tissue;
positioning the specimen modifying apparatus adjacent the tissue specimen to be modified; and
activating the tissue modifying apparatus to modify the tissue specimen and inhibit seeding from the tissue specimen.

2. The method according to claim 1, further comprising the step of retrieving the tissue specimen after modifying the specimen.

3. The method according to claim 1, further comprising the step of positioning an access port within the opening in tissue, the tissue modifying apparatus being inserted through the access port.

4. The method according to claim 3, further comprising the step of removing the specimen through the access port without encapsulating the specimen within a retrieval bag.

5. The method according to claim 3, wherein the access port includes:
a tubular body defining a longitudinal axis and configured for positioning within an opening in tissue, the tubular body having a proximal end and a distal end;
a proximal radial flange extending radially outwardly from the proximal end of the tubular body, the proximal radial flange configured to extend along an exterior surface of tissue adjacent the opening in tissue; and
a distal radial flange extending radially outwardly from the distal end of the tubular body, the distal radial flange configured to extend along an interior surface of tissue adjacent the opening in tissue.

6. The method according to claim 1, wherein the specimen modifying apparatus includes a housing having an elongated shaft extending from a distal end thereof, the shaft including a lumen therein for the passage of tissue sealant therethrough and configured for insertion through an opening in tissue.

7. The method according to claim 6, wherein the specimen modifying apparatus includes a dispensing assembly disposed at a distal end of the shaft and in communication with the lumen of the shaft, the dispensing assembly positionable adjacent a tissue specimen and configured to selectively dispense tissue sealant onto a surface of the tissue specimen to thereby seal the tissue specimen, inhibiting passage of cellular material between the tissue specimen and surrounding tissue.

8. The method according to claim 7, wherein the dispensing assembly includes a nozzle.

9. The method according to claim 1, wherein the specimen modifying apparatus includes a housing having an elongated shaft disposed at a distal end thereof, the housing being connected to a radiation source, the shaft being insertable through an opening in tissue.

10. The method according to claim 9, wherein the specimen modifying apparatus includes an end effector assembly disposed at a distal end of the shaft, the end effector assembly being positionable adjacent a tissue specimen and including a pair of insulated cups moveable from an open position to a closed position wherein the cups form a closed cavity in the closed position to thereby retain the tissue specimen therein, the cups being insulated to inhibit the passage of radiation therethrough; and
wherein, upon activation, radiation energy is supplied from the radiation source to the tissue specimen retained within the cups when in the closed position to terminate the tissue specimen.

11. The method according to claim 10, wherein movement of the cups to the closed position forms a spherical cavity.

12. The method according to claim 1, wherein the specimen modifying apparatus includes a housing having an elongated shaft disposed at a distal end thereof and configured to operably couple to a source of electrical energy.

13. The method according to claim 12, wherein the specimen modifying apparatus includes an end effector assembly disposed at a distal end of the shaft and being positionable at least partially through an opening in tissue, the end effector assembly including a pair of electrically conductive members spaced apart from one another and configured for positioning with a tissue specimen disposed therebetween, wherein electrical energy is selectively applicable from the electrical energy source to the pair of electrically conductive members to thereby pass a voltage through the specimen to modify the tissue specimen.

14. A specimen modifying apparatus comprising:
a housing having an elongated shaft extending from a distal end thereof, the shaft including a lumen therein for the passage of tissue sealant therethrough and configured for insertion through an opening in tissue; and
a dispensing assembly disposed at a distal end of the shaft and in communication with the lumen of the shaft, the dispensing assembly positionable adjacent a tissue specimen and configured to selectively dispense tissue sealant onto a surface of the tissue specimen to thereby seal the tissue specimen, inhibiting passage of cellular material between the tissue specimen and surrounding tissue.

15. The apparatus according to claim 14, wherein the dispensing assembly includes a nozzle.

16. A specimen modifying apparatus comprising:
a housing having an elongated shaft disposed at a distal end thereof, the housing being connected to an energy source, the shaft being insertable through an opening in tissue; and
an end effector assembly disposed at a distal end of the shaft, the end effector assembly being positionable adjacent a tissue specimen and including a pair of members moveable from an open position to a closed position;
wherein, upon activation energy is supplied from the energy source to the tissue specimen retained within the cups to modify the tissue specimen for removal from a body.

17. The apparatus according to claim 16, wherein the energy source is a source of radiation and the end effector assembly includes a pair of insulated cups which form a closed cavity in the closed position to thereby retain the tissue specimen therein, the cups being insulated to inhibit the passage of radiation therethrough.

18. The apparatus according to claim 17, wherein movement of the cups to the closed position forms a spherical cavity.

19. The apparatus according to claim 16, wherein the energy source is a source of electrical energy, and the end effector assembly includes a pair of electrically conductive plates spaced apart from one another and configured for positioning with a tissue specimen disposed therebetween, wherein electrical energy is selectively applicable from the electrical energy source to the pair of electrically conductive plates to thereby pass a voltage through the specimen to modify the tissue specimen.
